(19) United States
(12) Patent Application Publication
(10) Pub. No.: US 2017/0265750 A1
(43) Pub. Date: Sep. 21, 2017

(54) INFORMATION PROCESSING SYSTEM AND DISPLAY CONTROL METHOD

(71) Applicant: CANON KABUSHIKI KAISHA, Tokyo (JP)

(72) Inventors: Naoya Iizuka, Yokohama-shi (JP); Takuro Miyasato, Tokyo (JP); Kenichi Nagae, Yokohama-shi (JP)

(21) Appl. No.: 15/447,405

(22) Filed: Mar. 2, 2017

(30) Foreign Application Priority Data
Mar. 15, 2016 (JP) 2016-051074

(51) Int. Cl. A61B 5/00

(52) U.S. Cl. CPC A61B 5/0095; A61B 5/7425; A61B 5/748; A61B 2576/00

(57) ABSTRACT

There is employed an information processing system including: a photoacoustic image acquiring unit that acquires photoacoustic image data derived from photoacoustic waves generated by an object that is irradiated with light; a setting unit that sets a simulation condition; a reference image acquiring unit that acquires simulation image data being photoacoustic image data corresponding to the simulation condition set by the setting unit; and a display controlling unit that causes a photoacoustic image based on the photoacoustic image data and a reference image based on the simulation image data, to be displayed on a display unit.

### BACKGROUND OF THE INVENTION

Field of the Invention

The present invention relates to an information processing system and to a display control method.

Description of the Related Art

Photoacoustic imaging is one imaging technology that utilizes light. In photoacoustic imaging, firstly an object is irradiated with pulsed light generated by a light source. The irradiated light propagates and diffuses within the object. Acoustic waves (hereafter referred to as photoacoustic waves) are generated, on account of the photoacoustic effect when light energy is absorbed at a plurality of sites within the object. These photoacoustic waves are received by an ultrasound probe (transducer) and received signals are analyzed in a processing device, whereby information relating to optical characteristic values in the interior of the object is acquired in the form of image data (US Patent Application Publication No. 2013/0217995 (Specification)). An optical characteristic value distribution within the object is visualized accordingly. Recent years have witnessed rapid advances in pre-clinical research that involves capturing blood vessel images of small animals, relying on photoacoustic imaging, and in clinical research where these principles are used in the diagnosis of breast cancer or the like.

Display technologies of clinical images in ultrasound diagnosis devices include technologies where a reference image is displayed near an ultrasound image acquired by a device, in order to assist appropriate diagnosing. Reference images include for instance images having been acquired by the device in the past, and diagnosis images of another modality.

Patent Literature 1: US Patent Application Publication No. 2013/0217995

### SUMMARY OF THE INVENTION

The shape of the photoacoustic images visualized using a photoacoustic imaging apparatus vary depending on the thickness, position, angle and so forth of the observation target (for instance, blood vessels). For instance, an observation target having a cylindrical shape may be visualized in the form of a tubular shape having a cavity, or in the form of a virtual image referred to as an artifact. Such imaging characteristics often arise from device characteristics such as probe band, arrangement, reconstruction method, correction means and so forth. These imaging characteristics are often difficult to grasp intuitively, and hence interpretation by a user is difficult.

The present invention addresses the above problems. It is an object of the present invention to allow for better user interpretation during display of an image obtained through photoacoustic imaging.

The present invention provides an information processing system, comprising:

a photoacoustic image acquiring unit configured to acquire photoacoustic image data derived from photoacoustic waves generated from an object that is irradiated with light;

a setting unit configured to set a simulation condition;

a reference image acquiring unit configured to acquire simulation image data which is photoacoustic image data corresponding to the simulation condition set by the setting unit; and

a display controlling unit configured to cause a photoacoustic image based on the photoacoustic image data and a reference image based on the simulation image data, to be displayed on a display unit.

The present invention also provides an information processing system, comprising:

a photoacoustic image acquiring unit configured to acquire photoacoustic image data derived from photoacoustic waves generated from an object that is irradiated with light;

a reference image acquiring unit configured to acquire a plurality of pieces of simulation image data which are photoacoustic image data corresponding to a plurality of simulation conditions respectively;

a similarity degree acquiring unit configured to acquire a similarity degree between the plurality of pieces of simulation image data and the photoacoustic image data;

a determining unit configured to determine whether or not the similarity degree includes within a predefined numerical value range; and

a display controlling unit configured to cause a photoacoustic image based on the photoacoustic image data and the simulation condition corresponding to the simulation image data, the similarity degree of which has been determined by the determining unit to include within the predefined numerical value range, to be displayed on a display unit.

The present invention also provides a display control method, comprising the steps of:

displaying a photoacoustic image derived from photoacoustic waves generated from an object irradiated with light; and

displaying a reference image being a photoacoustic image obtained through simulation.

The present invention succeeds in allowing for better user interpretation during display of an image obtained through photoacoustic imaging.

Further features of the present invention will become apparent from the following description of exemplary embodiments with reference to the attached drawings.

### BRIEF DESCRIPTION OF THE DRAWINGS

FIG. 1 is a schematic diagram illustrating the overall configuration of a photoacoustic imaging apparatus of Embodiment 1;

FIG. 2A is a flowchart illustrating a process in Embodiment 1;

FIG. 2B is a flowchart illustrating a process in Embodiment 1;

FIG. 3 is a diagram illustrating an example of a display unit;

FIG. 4 is a schematic diagram illustrating the overall configuration of a photoacoustic imaging apparatus of Embodiment 2; and

FIG. 5 is a flowchart illustrating a process in Embodiment 2.

### BRIEF DESCRIPTION OF THE EMBODIMENTS

Preferred embodiments of the present invention will be explained next with reference to accompanying drawings.

The dimensions, materials and shapes of constituent parts, relative positions between the constituent parts, and other features are to be modified as appropriate in accordance with the configuration of the equipment to which the present invention is to be applied, and in accordance with various other conditions, and therefore do not constitute features that limit the scope of the invention to the disclosure that follows hereafter.

The present invention relates to a technology for detecting acoustic waves that propagate from an object, and for generating and acquiring characteristic information on the interior of the object. Accordingly, the present invention can be viewed as an object information acquiring apparatus or control method thereof, or as an object information acquisition method or a signal processing method, or as an information processing system. The present invention can further be viewed as a program for causing the foregoing methods to be executed in an information processing device provided with hardware resources, such as a CPU and the like, or as a storage medium in which such a program is stored.

The object information acquiring apparatus of the present invention encompasses apparatuses that reliably on the photoacoustic effect and in which light (electromagnetic waves) is irradiated onto an object, acoustic waves generated as a result within the object are received, and characteristic information on the object is acquired in the form of image data. Herein the term characteristic information denotes information on characteristic values corresponding to respective positions within the object and that are generated using received signals obtained through reception of photoacoustic waves.

The characteristic information acquired through photoacoustic measurement denotes values that reflect light energy absorptivity. Such characteristic information includes for instance a generation source of acoustic waves that are generated through light irradiation, initial sound pressure inside the object, or light energy absorption density or absorption coefficient derived from the initial sound pressure, as well as the concentration of tissue-constituting substances. Oxygen saturation distribution can be calculated by working out oxyhemoglobin concentration and deoxyhemoglobin concentration as substance concentrations. Glucose concentration, collagen concentration, melanin concentration and volume fractions of fat and water are also worked out herein.

A two-dimensional or three-dimensional characteristic information distribution is obtained on the basis of the characteristic information at each position within the object. Distribution data can be generated in the form of image data. The characteristic information may be worked out not as numerical value data but in the form of distribution information at each position within the object. Specifically, the characteristic information is distribution information such as an initial sound pressure distribution, an energy absorption density distribution, an absorption coefficient distribution or oxygen saturation distribution.

The term acoustic wave in the present invention encompasses typically ultrasonic waves, and includes elastic waves referred to as sound waves and acoustic waves. Electrical signals converted by a probe or the like from acoustic waves are also referred to as acoustic signals. The wavelength of these elastic waves is not meant to be limited by the disclosure pertaining to ultrasonic waves and acoustic waves in the present specification. Acoustic waves generated on account of the photoacoustic effect are referred to as photoacoustic waves or photoultrasonic waves. Electrical signals derived from photoacoustic waves are also referred to as photoacoustic signals.

A photoacoustic imaging apparatus that acquires object information by relying on the photoacoustic effect will be explained in the embodiments below. The main purpose of such a photoacoustic imaging apparatus include diagnosis of for instance vascular disease and malignant tumors, as well as chemotherapy follow-up, in humans and animals. In this case the object is part of a living body. A non-biological object such as a phantom may also serve as the object to be measured.

The present invention can be further viewed as a display control device for acquiring, from a memory or the like, an image having been acquired by a photoacoustic imaging apparatus, and presenting the image to a user, and as a display control method for presenting a photoacoustic image to a user.

### First Embodiment

### (System Configuration)

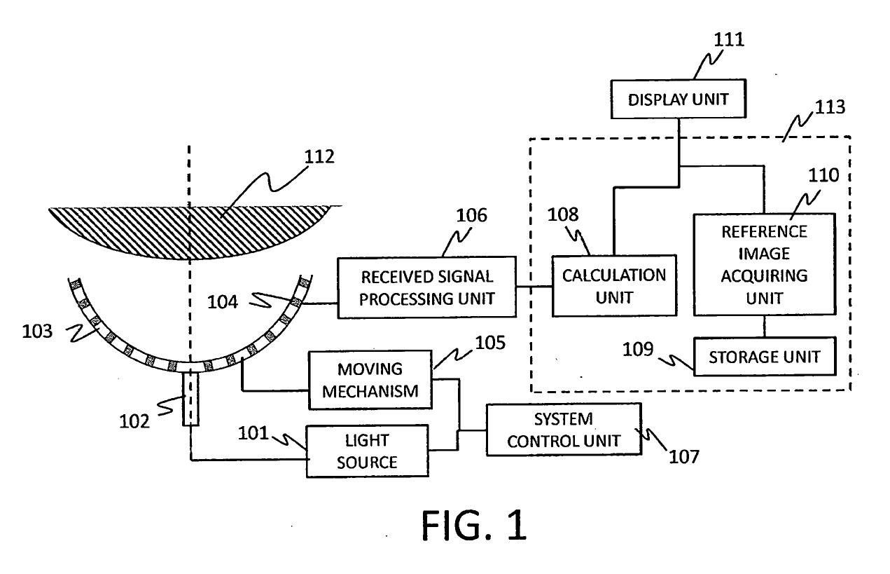

The schematic configuration of a photoacoustic imaging apparatus according to the present embodiment and specific examples of the various constituent elements will be explained next with reference to FIG. 1. A photoacoustic imaging apparatus is provided with a light source 101, an irradiation unit 102, a photoacoustic wave reception unit 103, a plurality of conversion elements 104 provided in the photoacoustic wave reception unit 103, a moving mechanism 105 for moving the photoacoustic wave reception unit 103, and a received signal processing unit 106. The photoacoustic imaging apparatus is further provided with a system control unit 107 that controls the light source 101 and the moving mechanism 105, a calculation unit 108 that reconstructs received signals to image data, and a storage unit 109 that stores image data from simulation. The photoacoustic imaging apparatus is further provided with at least a reference image acquiring unit 110 that generates a reference image from data in the storage unit, and a display unit 111. Characteristic information of an object 112 is acquired as a result. The calculation unit 108, the storage unit 109 and the reference image acquiring unit 110 may be present in an independent computer 113 that has a CPU, a main storage device and an auxiliary storage device, or may be custom-designed hardware. A workstation or the like is typically used herein.

(Light Source 101)

The light source is preferably a laser light source in order to obtain a large output. A light-emitting diode, a flash lamp or the like can also be used. Examples of lasers that can be used include for instance solid-state lasers, gas lasers, dye lasers, semiconductor lasers and the like. Ideally there can be used an Nd:YAG-pumped Ti:sa laser or an alexandrite laser having strong output and continuously tunable wavelength. Alternatively there may be used a plurality of single-wavelength lasers having dissimilar wavelengths.

The wavelength of the pulsed light is a specific wavelength that is absorbed by a specific component from among the components that make up the object, and is preferably a wavelength at which light propagates through the interior of the object. In the case of a biological object, specifically, the wavelength of the pulsed light includes preferably in the range of 700 nm to 1100 nm.

In order to effectively generate photoacoustic waves, light must be irradiated over a sufficiently short time in accordance with the thermal characteristics of the object. If the object is a biological one, the pulse width of the pulsed light generated by the light source includes preferably in the range of about 1 nanosecond to 100 nanoseconds. The pulsed light generated by the light source will be referred to hereafter as irradiated light.

(Irradiation Unit 102)

The irradiation unit is a means for guiding pulsed light emitted by the light source to the object, and irradiating the object with the pulsed light. The irradiation unit guides the irradiated light to the object while bringing the light to a desired irradiated light distribution shape. For instance a light-reflecting mirror, a light-magnifying lens, a light-diffusing diffusion plate or a waveguide such as an optical fiber can be used herein. Preferably, the light is spread over a certain surface area, from the viewpoint of safety towards the object and in terms of widening the diagnostic area.

(Photoacoustic Wave Reception Unit 103)

The photoacoustic wave reception unit 103 is a structure in which a plurality of conversion elements 104 is disposed on a bowl-shaped support. The elements are disposed in such a manner that there is formed a high-sensitivity region at which directions of high sensitivity (directional axes) are concentrated. The plurality of conversion elements 104 is disposed lined up in a three-dimensional spiral. The relative position with respect to the object is modified through movement of the plurality of conversion elements 104 along with the photoacoustic wave reception unit 103.

During photoacoustic measurement, a sound transmission medium (for instance, water or castor oil) is disposed between the photoacoustic wave reception unit 103 and the object 112. The photoacoustic imaging apparatus may be provided with a holding member (not shown) shaped as a thin cup, for holding the object 112. In this case a sound transmission medium is disposed also between the holding member and the object 112. Preferably, the holding member is a material that is highly transmissive towards light and acoustic waves, for instance polymethylpentene. The irradiation unit 102 that irradiates the object with light propagating from the light source 101 is provided below the photoacoustic wave reception unit 103. The irradiation unit 102 may however be provided separately from the photoacoustic wave reception unit 103.

(Conversion Elements 104)

The conversion elements 104 are a means for detecting acoustic waves generated within the object and for converting the acoustic waves to electrical signals. The conversion elements 104 are also referred to as probes, acoustic wave detectors or transducers. Acoustic waves generated by living bodies are typically ultrasonic waves having a frequency in the range of 100 kHz to 100 MHz. Accordingly, elements capable of detecting the above frequency band are used as the conversion elements 104. Specifically, there can be used transducers relying on piezoelectric phenomena, for instance of lead zirconate titanate (PZT or the like), transducers relying on light resonance and transducers relying on changes in electrostatic capacity such as CMUTs. Preferably, the conversion elements 104 have high sensitivity and a wide frequency band.

(Moving Mechanism 105)

The moving mechanism 105 moves the photoacoustic wave reception unit 103 to modify thereby the relative positions between the plurality of conversion elements 104 and the object 112. An automatic stage using a stepping motor or a servo motor can be used as the moving mechanism. The moving mechanism 105 may be a mechanism that moves the photoacoustic wave reception unit 103 in a two-dimensional spiral trajectory, or in a straight line, or in three dimensions.

(Reception Signal Processing Unit 106)

The received signal processing unit 106 has a means for amplifying the obtained electrical signals and converting the signals to a digital signal. Specifically, the received signal processing unit 106 may be an amplifier, an A/D converter, a FPGA chip or the like. In a case where the obtained received signal is a plurality of signals, it is preferable that these signals can be processed simultaneously. The image generation time can be reduced as a result. The acoustic wave signals detected at a same position with respect to the object may be integrated into one signal. The integration method resorted to herein may involve summation of signals, calculation of an average value or summation of weighted signals. In the present specification the term “received signal” encompasses conceptually both analog signals output by a photoacoustic wave reception unit as well as digital signals resulting from subsequent A/D conversion.

(Calculation Unit 108)

The calculation unit 108 is a means for processing a signal having undergone digital conversion, and for reconstructing an image that represents optical characteristics and morphological information of the interior of the object. Any method can be resorted to herein as the reconstruction method, for instance Fourier transformation, universal back projection, filtered back projection, phasing addition and the like. Characteristic information on the interior of the object is acquired in the form of a set of voxel data, when three-dimensional information is to be acquired, and as a set of pixel data when two-dimensional information is to be acquired. The generated image is transmitted, as photoacoustic image data, to the display unit 111, and is presented to the user. The calculation unit corresponds to the photoacoustic image acquiring unit of the present invention.

(Storage Unit)

The storage unit 109 is a storage device capable of storing electrical signals (received signals), image data, programs and so forth. A memory such as a ROM, a RAM, a hard disk or the like ordinarily provided in the computer 113 can be used herein as the storage unit 109.

(Simulation)

A plurality of pieces of simulation image data reconstructed on the basis of the simulation signals are stored in the storage unit 109. The simulation image data is preferably stored in database format, using keys in the form of for instance conditions pertaining to the object, conditions based on the structure of the photoacoustic imaging apparatus, and measurement conditions. Methods for creating simulation image data include image reconstruction using simulation signals. The simulation signal is a signal calculated in accordance with the hardware configuration of the photoacoustic wave imaging apparatus and in accordance with the method for creating display data. The simulation signal is obtained through simulation of the received signals output by the conversion elements 104. The term hardware configuration denotes herein the number and scanning range of the conversion elements of the photoacoustic wave reception unit 103, as well as the frequency band and the size of the conversion elements and so forth. The simulation image data is calculated through reconstruction of the simulation signal reflecting apparatus-specific characteristics.

Herein follows a more detailed example of the method for calculating simulation image data. An absorber model for imaging through simulation is set first. As the absorber model there can be set for instance a simple shape such as a sphere or a cylinder, or a more complex shape that imitates vascular structure, or a shape of new blood vessels around a tumor. The set absorber model is divided into a plurality of reconstruction units (voxels in the explanation hereafter but pixels in the case of two dimensions).

Received signals at the time of reception, by the conversion elements 104, of the acoustic waves generated by the plurality of voxels in the absorber model are computed next on the basis of the speed of sound and the length of the propagation path of acoustic waves at a time where the voxels are taken as an initial sound source. For example, the received signals can be calculated easily by assuming that each voxel is a spherical sound source in which light is absorbed uniformly, and by using an analytical solution. The characteristics of the conversion elements (element size, reception band and so forth) are preferably taken into account.

Next, characteristic information (for instance initial sound pressure) of the voxels corresponding to the position of the sound source at which the sound waves are generated is calculated using the computed received signals. In such image reconstruction it is preferable to use the same method as that during the actual photoacoustic measurement. Simulation image data can be acquired as a result. It is preferable to check the consistency of the simulation image data thus acquired with the photoacoustic image that is reconstructed on the basis of the actual photoacoustic measurement, and to carry out corrections as needed. The generated plurality of pieces of simulation image data are stored in the storage unit. The plurality of pieces of simulation image data may be stored beforehand, prior to calculation of the photoacoustic image of the object; alternatively, a photoacoustic image of the actual object may be checked, followed by generation of image data through simulation calculation using that information, and storage of the generated data. Image data may be calculated in accordance with parameters and simulation conditions, depending on the computing power.

(Reference Image Acquiring Unit)

In the reference image acquiring unit a reference image for display near the actual photoacoustic image of the object is generated using simulation images stored in the storage unit. The reference image is a juxtaposition of a plurality of images extracted from the storage unit, on the basis of image parameters that are set for instance through designation by the user. The term image parameters denote for instance the type, size, position (relative distance to the photoacoustic wave reception unit) and angle of the absorber model shape, as well as number of images, image line-up sequence and so forth. The reference image is not limited to being a juxtaposition of a plurality of images, and may be a single simulation image. In a case where a plurality of simulation images is used as the reference image there is utilized, as appropriate, a plurality of simulation images resulting from modifying a specific image parameter little by little.

The reference image is used in order to assist in the interpretation of the photoacoustic image that is obtained as a result of the actual photoacoustic measurement. The reference image is a simulation image taking into account device characteristics. The reference image indicates the specific manner in which the set absorber model is to be displayed as a photoacoustic image in the apparatus. By looking at the reference image, the user can judge comprehensively the specific absorber from which the image displayed on the display unit derives.

Further, an actual photoacoustic measurement may be performed on a real object or on a phantom having an artificial absorber embedded therein as a sound source, whereupon the received signal obtained as a result is subjected to image reconstruction, and is then stored in the storage unit together with the measurement conditions.

The calculation unit 108, the storage unit 109 and the reference image acquiring unit 110 can be realized for instance in the form of functional modules of the computer 113. The computer 113, which is provided with resources including a processor such as a CPU or GPU, a memory such as a ROM or RAM, a communication device, a user interface and so forth, is an information processing device that operates in accordance with the various steps of a program. The functions of the calculation unit 108, the storage unit 109 and the reference image acquiring unit 110 may be realized by combining a plurality of devices. The user interface may be an input device through which the user inputs information, for instance a mouse, a keyboard, a touch panel or the like, or a notifying device relying on sound or images.

(Display Controlling Unit)

The computer 113 functions as a display controlling unit for displaying images on the display unit 111. The display controlling unit causes a photoacoustic image based on the photoacoustic image data that is acquired through photoacoustic measurement of the object, to be displayed on the display unit. The display controlling unit further causes to be displayed, on the display unit, a reference image based on simulation image data derived from photoacoustic waves that would be generated upon irradiation of light onto the absorber model that imitates an absorber within the object, as a result of simulation under given simulation conditions. The display controlling unit further causes the absorber model used for simulation to be displayed, as a simulation model, on the display unit. The display functions ordinarily present in a computer can be resorted to herein for such display control.

(Display Unit 111)

The display unit 111 displays a photoacoustic image obtained through photoacoustic measurement, and a reference image generated through simulation. Any display device, such as a liquid crystal display (LCD), cathode ray tube (CRT), organic EL display or the like can be used as the display unit 111. The display unit may be integrated with the photoacoustic imaging apparatus, or may be provided separately.

Process relating to reception of photoacoustic waves

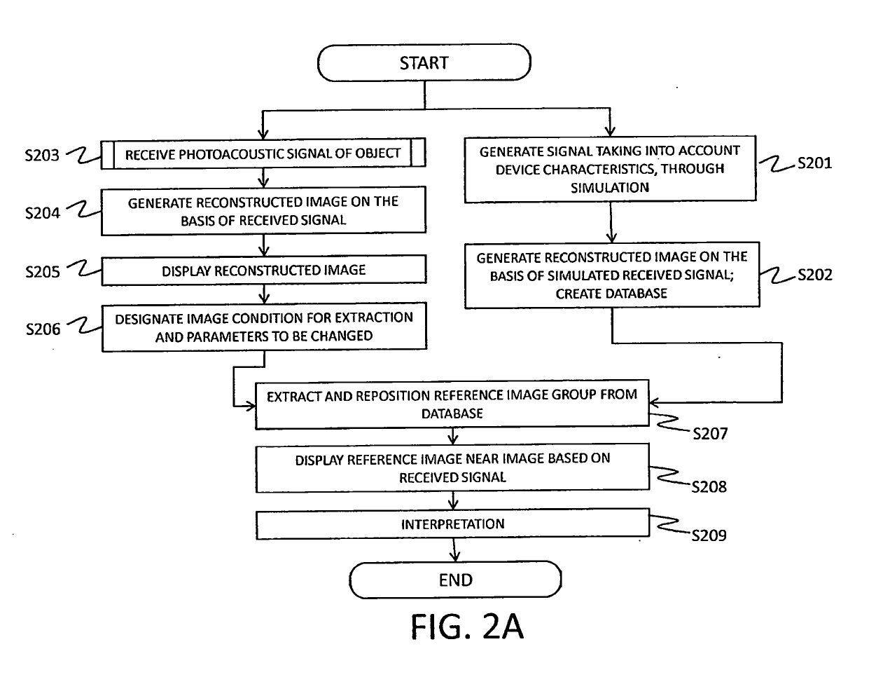

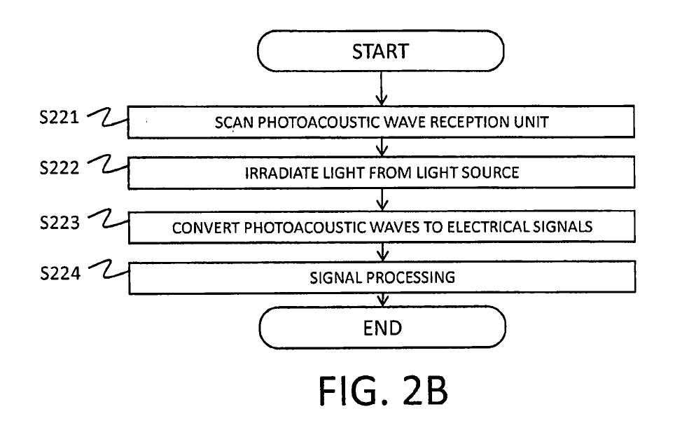

The process flow relative to reception of photoacoustic waves will be explained next with reference to FIG. 2B. This process explains in detail the flow performed in step S203 of FIG. 2A.

Firstly, the moving mechanism 105 moves the photoacoustic wave reception unit 103 along a predefined trajectory, in accordance with an instruction from the system control unit 107 (step 221). The light source 101 generates light at predefined emission intervals, in accordance with an instruction from the system control unit 107 (step 222). At a given timing during the movement of the photoacoustic wave reception unit 103, pulsed light generated by the light source 101 passes through the irradiation unit 102 and is irradiated onto the object 112. The propagation speed of light is sufficiently high, and accordingly the point in time of light emission by the light source 101 and the point in time at which the object is irradiated with that light can be regarded as identical.

Part of the energy of light that propagates through the interior of the object is absorbed by a light absorber (for instance, blood vessels having a significant amount of hemoglobin) that absorbs a predefined wavelength, whereupon photoacoustic waves are generated on account of thermal expansion of the light absorber. The photoacoustic wave reception unit 103 receives the photoacoustic waves and converts the photoacoustic waves into time-series received signals (step 223). The propagation speed of acoustic waves (speed of sound) is sufficiently higher than the movement speed of the photoacoustic wave reception unit 103. For convenience, therefore, the detection position at which a given conversion element receives the photoacoustic waves and the position that the conversion element occupies at the timing where the object 112 is irradiated with the pulsed light that generates that photoacoustic waves (position at which the conversion element is present) may be regarded as a same position. However, an image of yet higher precision can be generated, during image reconstruction, by performing a correction calculation based on the distance over which the photoacoustic wave reception unit 103 has moved since irradiation of the pulsed light until reception of the photoacoustic waves.

The light source 101 emits light at predefined periods, and the photoacoustic wave reception unit 103 moves at a predefined speed; at a point in time of light irradiation other than a previous light irradiation time, therefore, each conversion element 104 receives the photoacoustic waves at a detection position that is different from the detection position of the previous light irradiation time. The received signals output from the plurality of conversion elements 104 are sequentially input to the received signal processing unit 106, at respective reception timings (S224). The received signal processing unit 106 amplifies the received signals, subjects the resulting signals to AD conversion, and transmits the digitized received signals to the calculation unit 108. The received signals are stored in the storage unit for later information processing.

Process Relating to Image Display

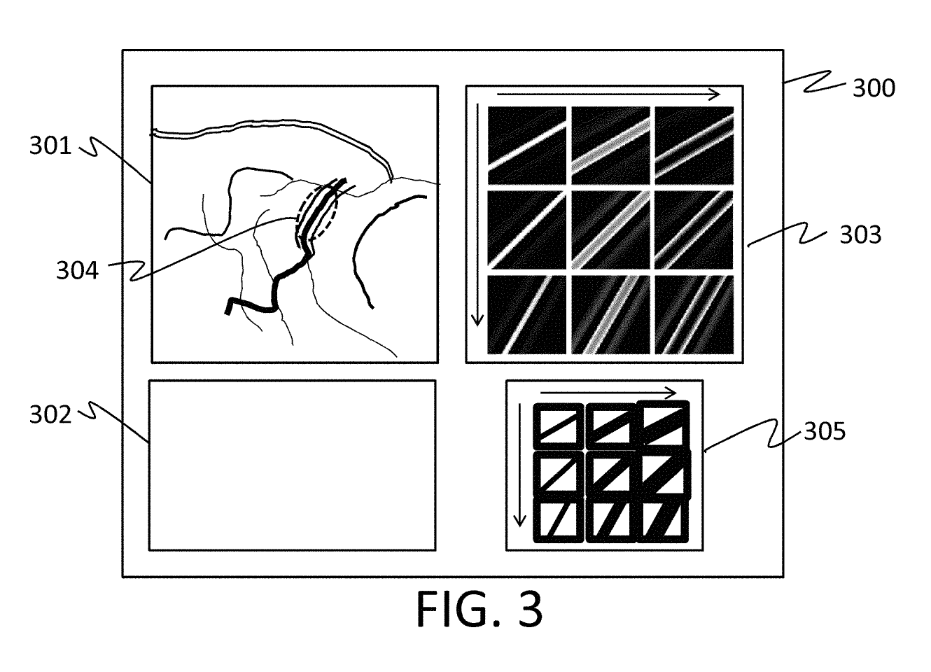

The process flow up to image display in the present embodiment will be explained next with reference to FIGS. 2A and 3. FIGS. 2A and 2B are flowcharts each illustrating a display method of a photoacoustic image and a reference image. FIG. 3 is a diagram illustrating an example of the display unit 111.

Step S201 is a step of generating a simulated received signal derived from simulation. In this step, there is generated a simulation signal resulting from simulating the received signals that are received and output by the actual conversion elements, through simulation taking into consideration, for instance, device characteristics such as conversion element shape, sensitivity frequency characteristic of the conversion elements, and information on the position of the conversion elements. This step is performed a plurality of times for a plurality of envisaged absorber models.

Step S202 is a step of reconstructing the simulated received signal to generate simulated photoacoustic image data (simulation image data), and making the generated image data into a database. As a result it becomes possible to acquire photoacoustic image data that represents optical characteristics and morphological information of the respective absorber model. This step is performed a plurality of times corresponding to the plurality of simulated received signals for a plurality of envisaged absorber models. Given the time required for calculations, steps S201 to S202 are preferably executed beforehand, prior to measurement of the object.

Step S203 is a step of receiving photoacoustic signals from the object and transmitting digitized received signals to the calculation unit, as described in the explanation of FIG. 2B. Subsequent step S204 is a step of reconstructing the received signals into photoacoustic image data. In this step, the calculation unit performs image reconstruction on the digital received signal having been received from the received signal processing unit, to generate photoacoustic image data that represents optical characteristics and morphological information of the interior of the object, and transmits the generated photoacoustic image data to the display unit. Step S205 is a step of displaying the photoacoustic image data on the display unit. In this step, the photoacoustic image data, which is object information received from the calculation unit, is displayed on the display unit. A shape of for instance a blood vessel, as a light absorber present inside the object, is displayed in this case.

FIG. 3 is a display example of the display unit. A display screen 300 has a photoacoustic image display section 301 that displays an image based on generated photoacoustic image data, and has, in addition, a reference image condition display section 302, a reference image display section 303 and a simulation model display section 305.

Step S206 is a step of designating a condition of the reference image that is to be displayed. In this step, there are designated the parameters that are necessary for establishing a reference image corresponding to a site that merits special attention, within the photoacoustic image that is displayed on the photoacoustic image display section 301. The designation method herein involves for instance manual input from a user by way of an input unit. Parameters that are necessary to establish the reference image include for instance type, size, position (relative distance to a sensor) and angle of the shape, as well as number of images and image line-up sequence. The parameters that are input herein are displayed on the reference image condition display section 302.

Generation of a reference image will be explained next using a concrete example. As an example, an instance will be explained where focus is laid on a blood vessel in a region of interest 304, within the photoacoustic image displayed on the photoacoustic image display section 301 illustrated in FIG. 3. Firstly, the type of the shape is designated in order to display a reference image for comparison with a blood vessel image of interest. For instance a sphere, a cylinder, a curved cylinder, a branch structure or a combination of the foregoing is prepared as the type of shape. Other than that, any shape may be used so long as it is useful in order to determine the shape of the target to be imaged in the photoacoustic imaging apparatus. Herein a cylindrical shape is designated in order to perform a comparison with a straight blood vessel.

Next there are designated the diameter and length of the cylinder, and the inclination angle and the position of the cylinder with respect to the bowl-shaped sensor. In case of display of a reference image made up of a plurality of simulation images in which specific parameters are changed little by little, there are designated the parameters that are to vary, and the ranges of the parameters. The ranges of the parameters that are caused to vary may be designated by the user; alternatively, there may be automatically set the ranges of change present beforehand in the reference image acquiring unit as default values. The designated reference image designation parameters are displayed on the reference image condition display section 302. Herein there is designated a cylindrical shape having a diameter of 2 mm and a length of 60 mm. Diameter and angle are set as the parameters that are to vary. There are designated three types of range of change of diameter, in 1 mm increments from 1 mm to 3 mm, and three types of range of change of angle, in 15° increments from 30° to 60°.

Step S207 is a step of extracting and repositioning reference images from a simulation image database. In this step, a simulation condition corresponding to the parameters for display of the reference image as designated in S206 are set in the reference image acquiring unit, and simulation images corresponding to the simulation condition are extracted from an image database. The reference image acquiring unit functions herein as the setting unit of the present invention. The reference images for the designated condition are generated and are disposed in order on the screen. In S206, there are designated three respective types of diameters and angles, and accordingly nine types of simulation images are extracted from the database and made into reference images. In the present example also an image of an absorber model corresponding to the simulation images is extracted and made into a simulation model images.

Step S208 is a step of displaying the reference images generated in S207 near the photoacoustic image that has been displayed in S205. The reference images are displayed on the reference image display section 303 at the top right in the figure. The absorber model corresponding to the simulation images is displayed on the simulation model display section 305 at the bottom right in the figure. If the reference image is to be modified it suffices to return to step S206 and re-set the parameters. The display method of images is not limited to adjacent display. For instance, the reference images may be displayed superimposed on the photoacoustic image. Alternatively, the photoacoustic image and the reference images may be displayed on separate displays.

Step S209 is a step of performing interpretation using the photoacoustic image displayed on the photoacoustic image display section 301 and the reference image group displayed on the reference image display section 303. As illustrated in FIG. 3 a plurality of reference images of a condition resembling the blood vessel of the region of interest 304 is displayed on the reference image display section 303. These reference images are acquired through reconstruction of simulation signals that reflect system characteristics of the apparatus. This makes therefore clear the specific manner in which the designated absorber model is to be displayed on the apparatus. The user can correctly judge object shapes by comparing the reference images and the absorber model with a region of interest. Alternatively, the user need not watch the images, and the information processing device may instead perform automatic recognition based on the photoacoustic image data and a reference image data group.

Herein the blood vessel of interest that is actually measured is flanked on both sides by visible lines of comparatively low intensity that run alongside the blood vessel of interest, as in the region of interest 304 in FIG. 3. In the absence of reference images the user may in some instances construe these lines as a blood vessel. By referring to the reference images displayed on the reference image display section 303, however, the user can grasp that the above lines are the result of imaging of a virtual image that traces the cylindrical absorber, for a specific diameter and a specific angle. This is an artifact caused by the arrangement of elements in the apparatus sensors. It is difficult for the user to grasp all appearance patterns of such artifacts. By displaying the reference images near the actually measured photoacoustic image it becomes possible to derive a more comprehensive interpretation and judgment in which device characteristics have been taken into account.

### Second Embodiment

A process in an object information acquiring apparatus of a second embodiment will be explained next. The configuration of the photoacoustic imaging apparatus of the present embodiment is basically identical to that of Embodiment 1. The explanation below will focus on differences with respect to Embodiment 1.

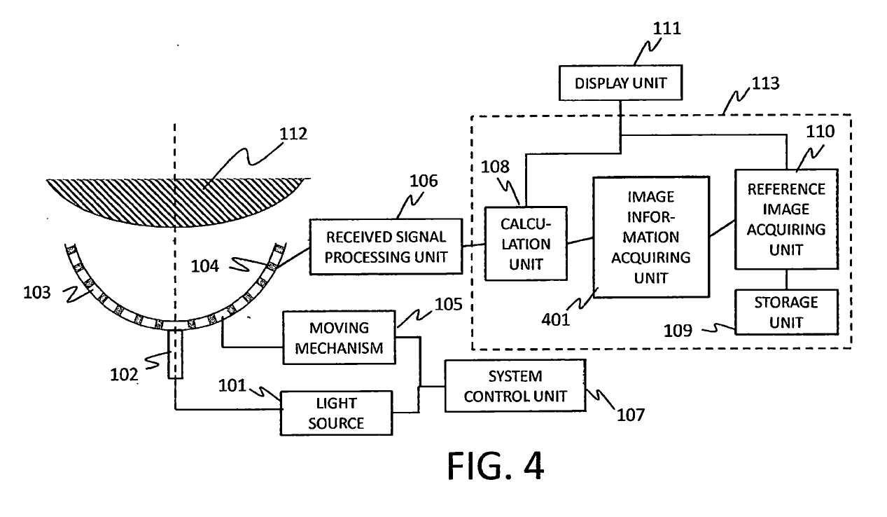

FIG. 4 illustrates the configuration of the apparatus of the present embodiment. In Embodiment 1 the region of interest and the reference image parameters are designated by a user who is viewing the photoacoustic image. In the present embodiment, on the other hand, photoacoustic image data calculated by a calculation unit is transmitted to an image information acquiring unit 401, and designation parameters of the reference images are established in the image information acquiring unit 401.

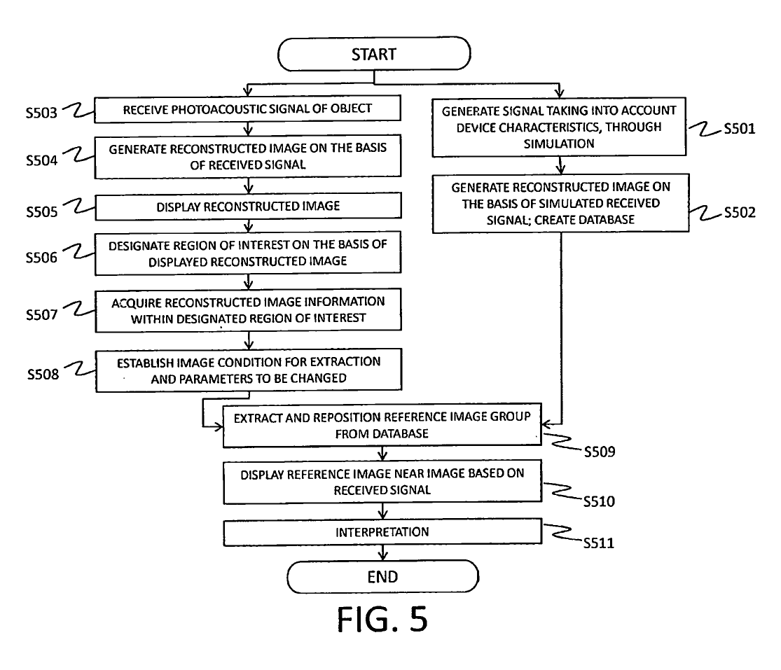

FIG. 5 is a diagram illustrating a process flow relating to image display in the present embodiment. The process from step S501 to S505 is identical to the process from S201 to S205 in Embodiment 1. Step S506 is a step of designating a region of interest on the basis of a photoacoustic image acquired by the apparatus. As illustrated in FIG. 3, there is designated a region of interest 304 that includes an observation target of interest, from within a photoacoustic image that is displayed on the photoacoustic image display section 301. The region of interest is designated by way of an input unit such as mouse, a keyboard or the like. The computer 113 may extract automatically a region of interest. Alternatively, the computer 113 may extract a feature image automatically.

Step S507 is a step in which an image information acquiring unit 401 acquires image information within the region of interest. In the present step a feature image is extracted from within the region of interest, in accordance with an image processing method such as feature detection or threshold value processing, and shape information of the feature image is acquired. In the example of FIG. 3 the type of shape is a cylinder, and there is acquired shape information such as the position, size in terms of diameter and length, and arrangement angle of the cylinder. Herein, for instance, shape information indicating that the cylinder has a diameter of 2 mm and an angle of 45° is acquired. The term shape information encompasses conceptually also position information and arrangement information.

Step S508 is a step of establishing a parameter for designating reference images. The reference image acquiring unit establishes reference image parameters on the basis of the acquired shape information. Herein the reference image parameters are established on the basis of parameters such as the diameter and angle of the cylinder. Preferably there are established neighbor values of the parameters. Values designated via the input unit can be used as the original parameters. For instance, some methods involve selecting a value close to a designated value, from among a plurality of choices set beforehand at predefined intervals. The number of neighbor values is not limited.

The process from step S509 to S511 is identical to the process from S207 to S209 in Embodiment 1. Specifically, reference images are generated through extraction from the simulation image database and through repositioning, in accordance with the reference image parameters established in S508, and are displayed near the photoacoustic image acquired by the apparatus. The user performs interpretation while referring to the reference images.

The reference images generated on the basis of the shape information acquired by the image information acquiring unit 401 are displayed; thereafter, the reference image parameters are modified, and different reference images are generated once more and are displayed. The computer 113 may calculate, and display, a correspondence with the absorber shape within the region of interest, for each reference image. The computer 113 may select, and present, an image that is closest to the absorber with the region of interest, from among the reference images. The computer 113 may also present a simulation model that is closest to the actual absorber.

In the photoacoustic imaging apparatus according to the present embodiment, as explained above, a more comprehensive interpretation and judgment in which device characteristics are taken into account can be derived by displaying simulation images, as reference images, in the vicinity of the actually measured photoacoustic image.

(Variation)

The display unit need not necessarily display a simulation image or an image of an absorber model. For instance, there may be calculated the specific kind of absorber that is present in a region of interest designated by the computer, and shape information of the absorber be displayed on the display unit as character information and/or as numerals.

The computer may extract a feature image, and there may be acquired a similarity degree between the feature image and a plurality of simulation images acquired from a memory. In this case, interpretation by the user can be assisted through display, on the display unit, of simulation conditions and parameters corresponding to a simulation image for which the display controlling unit has determined a high similarity degree. A known image recognition technology such as feature value extraction or edge detection can be used to acquire the degree of similarity. The computer functions in this case as the similarity degree acquiring unit of the present invention. The computer functions also as the determining unit of the present invention. Specifically, the computer determines whether or not the similarity degree includes within a predefined numerical value range. On the display unit there is displayed a simulation condition corresponding to simulation image data, the similarity degree of which has been determined by the determining unit to include within a predefined numerical value range.

As described above, the present invention allows displaying, as a reference image, a simulation image based on imaging characteristics unique to an apparatus, near the image to be observed. The user can make a more comprehensive judgment in which device characteristics have been taken into account, by performing interpretation using a reference image as an index of the degree of reliability of the image to be observed.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2016-051074, filed on Mar. 15, 2016, which is hereby incorporated by reference herein in its entirety.

What is claimed is:

1. An information processing system, comprising:
a photoacoustic image acquiring unit configured to acquire photoacoustic image data derived from photoacoustic waves generated from an object that is irradiated with light;
a setting unit configured to set a simulation condition;
a reference image acquiring unit configured to acquire simulation image data which is photoacoustic image data corresponding to the simulation condition set by the setting unit; and
a display controlling unit configured to cause a photoacoustic image based on the photoacoustic image data and a reference image based on the simulation image data, to be displayed on a display unit.

2. The information processing system according to claim 1,
wherein the reference image acquiring unit is configured to acquire the simulation image based on photoacoustic image data derived from photoacoustic waves generated in a case where an absorber model is irradiated with light, under the simulation condition set by the setting unit.

3. The information processing system according to claim 2, further comprising an input unit configured to allow designation of information,
wherein the setting unit is configured to set the simulation condition on the basis of the information designated by the input unit.

4. The information processing system according to claim 3, further comprising an input unit configured to allow designation of a parameter of the absorber model,
wherein the setting unit is configured to set the simulation condition on the basis of the parameter of the absorber model designated by the input unit, and
the reference image acquiring unit is configured to acquire the simulation image data under the simulation condition.

5. The information processing system according to claim 4,
wherein the parameter includes a parameter relating to a shape of the absorber model.

6. The information processing system according to claim 5,
wherein the parameter relating to the shape includes a type, a size and an arrangement angle of the shape of the absorber model.

7. The information processing system according to claim 1, further comprising an input unit configured to allow designation of a region of interest within the photoacoustic image,
wherein the setting unit is configured to set the simulation condition on the basis of an image within the region of interest designated by the input unit, and
the reference image acquiring unit is configured to acquire the simulation image data under the simulation condition.

8. The information processing system according to claim 1,
wherein the setting unit is configured to extract a feature image from the photoacoustic image data and set the simulation condition on the basis of the feature image.

9. The information processing system according to claim 1,
wherein the setting unit is configured to set a plurality of simulation conditions;
the reference image acquiring unit is configured to acquire a plurality of pieces of simulation image data corresponding to the plurality of simulation conditions; and
the display controlling unit is configured to cause the photoacoustic image and a plurality of reference images based on the plurality of pieces of simulation image data, to be displayed on the display unit.

10. The information processing system according to claim 1, further comprising a memory in which the simulation condition is stored,
wherein the setting unit is configured to perform setting by reading the simulation condition from the memory.

11. The information processing system according to claim 1, further comprising a memory in which a plurality of pieces of the simulation image data are stored,
wherein the reference image acquiring unit is configured to acquire the simulation image data corresponding to the simulation condition set by the setting unit, by reading the simulation image data from among the plurality of pieces of simulation image data stored in the memory.

12. The information processing system according to claim 1,
wherein the display controlling unit is configured to cause the photoacoustic image and the reference image to be displayed on the display unit through adjacent display or superimposed display.

13. An information processing system, comprising:
a photoacoustic image acquiring unit configured to acquire photoacoustic image data derived from photoacoustic waves generated from an object that is irradiated with light;
a reference image acquiring unit configured to acquire a plurality of pieces of simulation image data which are photoacoustic image data corresponding to a plurality of simulation conditions respectively;
a similarity degree acquiring unit configured to acquire a similarity degree between the plurality of pieces of simulation image data and the photoacoustic image data;
a determining unit configured to determine whether or not the similarity degree includes within a predefined numerical value range; and
a display controlling unit configured to cause a photoacoustic image based on the photoacoustic image data and the simulation condition corresponding to the simulation image data, the similarity degree of which has been determined by the determining unit to include within the predefined numerical value range, to be displayed on a display unit.

14. The information processing system according to claim 13, further comprising an input unit configured to allow designation of a region of interest within the photoacoustic image,
wherein the similarity degree acquiring unit is configured to acquire, as the similarity degree, a degree of similarity between the photoacoustic image within the region of interest designated by the input unit and the plurality of pieces of simulation image data.

15. A display control method, comprising the steps of:
displaying a photoacoustic image derived from photoacoustic waves generated from an object irradiated with light; and
displaying a reference image being a photoacoustic image obtained through simulation.

16. The display control method according to claim 15, further comprising the step of receiving a designation of a simulation condition,
wherein the step of displaying simulation image data includes a step of displaying simulation image data corresponding to the designated simulation condition.
